(19) United States
(12) Patent Application Publication
(10) Pub. No.: US 2026/0137754 A1
(43) Pub. Date: May 21, 2026

(54) APPLICATION OF A COMBINATION DRUG IN THE TREATMENT OF PATHOGENIC BACTERIAL PERSISTENT INFECTIONS

(71) Applicant: NOVOHEALTH (SUZHOU) BIOTECH CO., LTD., Suzhou (CN)

(72) Inventors: Yuzhi HONG, Suzhou (CN); Yanghui YE, Suzhou (CN); Mingxin DUAN, Suzhou (CN); Yuanqing TIAN, Suzhou (CN)

(21) Appl. No.: 19/450,280

(22) Filed: Jan. 15, 2026

Related U.S. Application Data
(63) Continuation of application No. PCT/CN2023/142138, filed on Dec. 26, 2023.

(51) Int. Cl. A61K 38/12; A61K 31/7036; A61K 31/7048; A61K 45/06; A61P 31/04

(52) U.S. Cl. CPC A61K 38/12; A61K 31/7036; A61K 31/7048; A61K 45/06; A61P 31/04

(57) ABSTRACT

Disclosed is the use of a combined compound medicine in treating pathogenic bacteria persistent infection. Existing antibiotic monotherapy or combination therapy is usually difficult to kill pathogenic bacteria in the persister state, especially nutrient-insensitive persister bacteria, which often lead to a series of problems of long treatment period, frequent condition recurrence and deterioration, treatment failure, drug-resistant mutation, etc. At present, drugs capable of efficiently killing highly persistent pathogenic bacteria in a clinically applicable concentration range are still unavailable. The novel compound medicine of polymyxin and aminoglycoside antibiotics disclosed in the present disclosure without precedence is capable of destroying bacterial cell membranes in a short time within a relatively wide range of clinically achievable plasma concentrations, leading to rapid death of a large number of bacteria, but is safe for human-derived cells.

### TECHNICAL FIELD

The present invention belongs to the field of biomedical technology and relates to the application of a combination drug in the treatment of pathogenic bacterial persistent infections, specifically to the use of a combination compound drug comprising polymyxin and aminoglycoside antibiotics in the treatment of pathogenic bacterial persistent infections.

### BACKGROUND

Bacterial persistence accounts for nearly half of clinical antibiotic treatment failures, and it is the main cause of chronic infections, recurrent and worsening of the disease, and can accelerate the generation of drug resistance mutations, further exacerbating the crisis of antibiotic resistance.

The mechanisms of bacterial persistence have seen some progress over the past two decades, yet remain controversial and not fully elucidated. Based on the presence or absence of sufficient exogenous nutrients in bacterial cultures during antibiotic treatment, reported in-vitro persistence models can be divided into two types: (1) “Nutrient-insensitive” persistence. Bacterial cells exhibit strong tolerance to nearly all bactericidal antibiotics even under abundant exogenous nutrients and they survive even when exposed to high antibiotic doses, which is a phenomenon termed “nutrient-insensitive” persistence. This represents true persistence and is the type the invention aims to overcome. (2) “Nutrient-sensitive” persistence/tolerance. This state, previously mistaken for persistence, occurs when nutrient deficiency in the culture prevents antibiotics from reaching their targets or generating sufficient reactive oxygen species, resulting in minimal cell death. Based on its definition and mechanism, the survival phenotype should actually be categorized as tolerance rather than true persistence. The “persistence” in most literature reports falls into this type. These models typically involve directly adding antibiotics to bacterial cultures during the stationary phase (without fresh exogenous nutrients) or treating starving bacterial cells in physiological saline. Surviving bacteria can often be completely killed and cleared under adequate nutrient conditions.

The clinical anti-infection therapies lack effective drugs and methods to eliminate “nutrient-insensitive” persistent pathogens. Current reports on drug-mediated elimination of persistent bacteria primarily rely on the aforementioned second type of “nutrient-sensitive” persistent/tolerant models. In 2011, Professor James Collins'team at MIT demonstrated that directly adding glucose, fructose, or mannitol to LB liquid culture of wild-type Escherichia coli during the stationary phase enhanced gentamicin's bactericidal efficacy. This effect was completely inhibited by carbonyl cyanide picolylhydrazone (CCCP), a proton motive force inhibitor, indicating that gentamicin's intracellular uptake depends on proton motive force. Similarly, Professor Kim Lewis'team at Northeastern University reported that the antimicrobial peptide ADEP4 could continuously activate the protease ClpP, effectively killing stationary-phase Escherichia coli in vitro. However, clinical trials of this compound were unsuccessful. In 2023, Professor Fu Xinmiao's team reported that heat treatment of stationary-phase Escherichia coli cultures at 55° C. for five minutes enhanced the bactericidal effect of aminoglycosides on persistent bacteria. Unfortunately, this method cannot be applied to treating internal tissue infections as most human cells could not withstand heat shock. Additionally, the proton motive force inhibitor CCCP can be combined with high concentrations of Aminoglycosides (500 μg/mL), Ampicillin, or Ciprofloxacin to effectively eliminate stationary-phase bacteria. However, CCCP remains highly toxic to both human and animal cells even at lower concentrations, making it unsuitable for clinical use. In summary, conventional antibiotic monotherapy fails to achieve satisfactory clinical outcomes by eradicating infecting persistent bacteria, and no combination antibiotic therapy has been reported to effectively eliminate persistent bacteria at clinically achievable serum concentrations.

Aminoglycoside antibiotics alone fail to effectively eradicate “nutrient-insensitive” persistent bacteria. Aminoglycosides are a well-known group of antibiotics characterized by highly similar structures and bactericidal mechanisms, encompassing various functionally analogous antibiotic molecules. Common examples include Kanamycin, Streptomycin, Amikacin, Tobramycin, Gentamicin, and Netilmicin. Within clinically achievable serum concentration range, these antibiotics generally exhibit no bactericidal effect on such persistent bacteria.

The combined use of polymyxins with other bactericidal antibiotics for eliminating persistent bacteria has not been reported. Polymyxins, a well-known group of peptide antibiotics with highly similar molecular structures and bactericidal mechanisms, inhibit most Gram-negative bacteria. Structurally, they differ from other antibiotics primarily by containing an ester acyl group and two amino acids. Representative clinical agents primarily include Polymyxin B and Polymyxin E, which are currently employed as second-line antibiotics.

In summary, the discovery of novel molecules or combination formulations of existing antibiotics that can rapidly eradicate persistent bacteria is of great significance for clinical anti-infection treatment, especially for chronic infection and recurrent bacterial infection diseases, and for improving public health, as well as for generating great economic value in the biomedical field.

### TECHNICAL PROBLEMS

Conventional monotherapy or combination antibiotic regimens generally fail to eradicate persistent pathogens, particularly nutrient-insensitive persistent bacteria. These persistent bacteria often lead to prolonged treatment duration, disease recurrence and exacerbation, treatment failure, and drug resistance mutations. To date, no clinically effective drugs have been developed to efficiently eliminate highly persistent pathogens within clinically applicable concentrations. The novel polymyxin-aminoglycoside combination drug disclosed in this invention for the first time demonstrates rapid bacterial cell membrane disruption within clinically achievable serum concentrations, achieving rapid and massive pathogen elimination while ensuring safety of human cells. This breakthrough holds promise for treating persistent bacterial infections, potentially shortening treatment duration, preventing relapses, and slowing resistance development.

### TECHNICAL SOLUTIONS

The invention adopts the following technical solution:

The application of a combination drug in the preparation of a medicament for treating pathogenic bacterial persistent infections, and the combination drug comprises polymyxin antibiotics and an aminoglycoside antibiotics.

The application of a combination drug in the preparation of a medicament for inhibiting pathogenic bacterial persistence, and the combination drug comprises polymyxin antibiotics and aminoglycoside antibiotics.

The application of a combination drug in the preparation of a medicament to synergistically eliminate persistent pathogenic bacteria, and the combination drug comprises polymyxin antibiotics and aminoglycoside antibiotics. Further, the drug also comprises other antibacterial agents.

The combination drug of the invention effectively inhibits pathogenic bacterial persistence while maintaining safety for human cells. As common sense, “safety” refers to pharmaceutically acceptable safety. Human-derived THP-1 cells treated with the novel polymyxin-aminoglycoside combination antibiotic of the present invention showed no significant difference in growth and metabolism compared to the normal growth control group without antibiotics. In contrast, the conventional respiratory chain proton motive force inhibitor CCCP significantly inhibited growth and resulted in cell death. Therefore, the bactericidal peak blood concentration of the composite antibiotics in clinical use is substantially lower than the test concentrations mentioned above, theoretically ensuring safety for human-derived cells.

Preferably, the pathogenic bacterium is a nutrient-insensitive persister. Bacterial cells exhibit exceptional tolerance to nearly all bactericidal antibiotics even under abundant exogenous nutrients, remaining viable despite high-dose antibiotic exposure. This phenomenon, termed “nutrient-insensitive persistence,” represents authentic bacterial persistence. Current clinical anti-infective therapies lack effective agents and methods to eliminate these persistent pathogens. Existing technologies for targeting persisters primarily focus on nutrient-sensitive persistence/tolerance, while therapeutic strategies specifically against nutrient-insensitive persisters remain scarce. The present invention discloses for the first time a combination drug integrating polymyxins and aminoglycoside antibiotics, which rapidly eliminates inactive persisters in large quantities, demonstrating superior efficacy compared to monotherapy. It also outperforms individual antibiotics in combating actively metabolizing pathogens. These advantages highlight its significant clinical potential for treating bacterial infections including pneumonia, bloodstream infections, urinary tract infections, respiratory infections, and intestinal infections, with promising applications in in the treatment of bacterial infections in animals.

Therefore, the present invention discloses the application of a combination drug in the preparation of anti-inflammatory or anti-infective drugs, and the combination drug comprises polymyxin and aminoglycoside antibiotics.

In the present invention, the polymyxin antibiotic comprises polymyxin or its salt, hydrate, solvate; the aminoglycoside antibiotic comprises aminoglycoside or its salt, hydrate, solvate.

In the present invention, aminoglycoside antibiotics are a large class of molecules with similar structure and function. Aminoglycoside antibiotics include Kanamycin, Amikacin, Tobramycin, Gentamicin and Streptomycin commonly used in clinic, as well as other aminoglycoside drug molecules with similar structure and function.

In the present invention, polymyxins are a large class of molecules with similar structure and function, including Polymyxin B, Polymyxin E, and other molecules with similar structure and function.

In this invention, the content ratio of aminoglycoside antibiotics in the combination drug ranges from 0.01% to 99.9%, preferably 75% to 99%, and more preferably 85% to 99%. By weight percentage, the range may extend to 86% to 99%, 87% to 99%, 88% to 99%, 89% to 99%, 90% to 99%, 91% to 99%, 92% to 99%, 93% to 99%, 94% to 99%, 95% to 99%, 96% to 99%, 97% to 99%, and 98% to 99%. These ratios are calculated based on the total weight of “polymyxin antibiotics and aminoglycoside antibiotics” as 100%, excluding other components such as carriers. If other carrier components are included, the corresponding proportional calculations should be made according to their respective weights.

In the present invention, the active ingredients of the combination drug are polymyxin antibiotics and aminoglycoside antibiotics. Additionally, it may comprise a carrier. As common sense, the active components—polymyxin and aminoglycoside antibiotics—are housed in the same carrier, forming a compound drug known as the polymyxin-aminoglycoside compound antibiotic.

In the present invention, the pathogenic bacteria are in the stationary phase or exponential growth phase. The polymyxin-aminoglycoside combination antibiotic not only effectively eliminates persistent bacteria in stationary phase cultures but also efficiently kills cells in the exponential growth phase, reducing their survival rate by seven orders of magnitude and decreasing persistence levels by a factor of 100. Whether bacterial cells are in the metabolically active state of exponential growth or in the persisting state of slow metabolism or stagnation, this combination antibiotic demonstrates high efficacy in killing and eliminating them.

In the present invention, the pathogenic bacteria include Gram-negative and Gram-positive bacteria. The polymyxin-aminoglycoside combination antibiotic demonstrated 100-fold reduction in persistent survival levels in cultured clinical isolates of Escherichia coli and Klebsiella pneumoniae compared to Amikacin monotherapy group. For clinical isolates of Pseudomonas aeruginosa and Staphylococcus aureus, the persistent survival levels were reduced by at least 10-fold. These results indicate that the combination antibiotic outperforms existing monotherapies in eliminating persisters, showing universal effectiveness across both Gram-negative and Gram-positive bacteria.

### BENEFICIAL EFFECTS

Compared with the prior art, the present invention has the following advantages:

(1) The first disclosed combination drug of polymyxin and aminoglycoside antibiotics in this invention can rapidly and extensively kill inactive metabolizing persistent bacteria, demonstrating significantly better efficacy than monotherapy with individual antibiotics. It also outperforms individual antibiotics in eliminating actively metabolizing and proliferating bacteria. Other antibiotic combinations within clinical concentrations, including those with polymyxin and commonly used beta-lactam or quinolone antibiotics, have not shown substantial effects in rapidly eliminating nutrient-insensitive persistent bacteria. In this combination drug, the combination index of polymyxin B and E with aminoglycosides reaches 0.25 and 0.4 respectively, exhibiting rapid disruption of bacterial cell membrane function. Notably, neither polymyxin nor aminoglycosides alone at equivalent concentrations achieve comparable cell membrane disruption or persistent bacterial elimination. Therefore, this combination drug demonstrates significant clinical potential for treating bacterial infections, including but not limited to pneumonia, bloodstream infections, urinary tract infections, respiratory infections, and intestinal infections, as well as for treating bacterial infections in animals.

(2) The polycycline and aminoglycoside antibiotic combination drug disclosed in this invention demonstrates broad-spectrum efficacy against representative Gram-negative and Gram-positive bacteria. Its clinical applications in anti-infective therapy and animal studies extend far beyond the tested bacterial strains, making it applicable to the vast majority of pathogenic bacteria.

### DESCRIPTIONS OF THE DRAWINGS

FIG. 1 shows the survival rates of wild-type E. coli after exposure to different bactericidal antibiotics, with A for 3 MIC kanamycin (MIC=8 μg/mL), B for 20 MIC ciprofloxacin (MIC=0.03 μg/mL), C for 20 MIC ampicillin (MIC=6 μg/mL), and D for 3 MIC polymyxin B (MIC=0.3 μg/mL).

FIG. 2 shows the high persistence survival rate of the hipA7 strain against various bactericidal antibiotics, with the wild-type strain as control, with A for 3 MIC kanamycin, 3 MIC streptomycin, B for 20 MIC ciprofloxacin, 20 MIC ampicillin, and C for 3 MIC polymyxin B, 3 MIC mitomycin C (MIC=0.8μg/mL).

FIG. 3 shows the high persistence survival rate of the metG2 strain against different bactericidal antibiotics, with the wild-type strain as control with A for 3 MIC kanamycin, 20 MIC ciprofloxacin, 20 MIC ampicillin and B for 3 MIC polymyxin B, 3 MIC mitomycin C.

FIG. 4 shows the persistence survival rate of the hipA7 strain against various antibiotic combinations, with the wild-type strain serving as control, with A for 20 MIC ciprofloxacin+20 MIC ampicillin, B for 20 MIC ciprofloxacin+3 MIC kanamycin, C for 20 MIC ciprofloxacin+3 MIC streptomycin, D for 20 MIC ciprofloxacin+3 MIC polymyxin B, E for 20 MIC ciprofloxacin+3 MIC polymyxin E, F for 20 MIC ciprofloxacin+3 MIC mitomycin C, and G for 20 MIC ampicillin+3 MIC kanamycin.

FIG. 5 shows the high persistence survival rate of the metG2 strain against different bactericidal antibiotic combinations, with the wild-type strain as control, with A for 20 MIC ciprofloxacin+20 MIC ampicillin, B for 20 MIC ciprofloxacin+3 MIC streptomycin, C for 20 MIC ciprofloxacin+3 MIC polymyxin B, D for 20 MIC ciprofloxacin+3 MIC polymyxin E, E for 20 MIC ciprofloxacin+3 MIC mitomycin C, and F for 20 MIC ampicillin+3 MIC kanamycin).

FIG. 6 shows the highly effective eradication of hipA7 high-persister bacteria by the polymyxin B-kanamycin combination, with A for kanamycin (final concentration 20 μg/mL)-polymyxin B (0.9 μg/mL) combination drug and control group of 20 μg/mL kanamycin or 0.9 μg/mL polymyxin B as monotherapy; with B for the combination drug containing 20 μg/mL kanamycin with polymyxin B at 0.003, 0.03, 0.3, or 0.6μg/mL, respectively; or alternatively, a combination comprising 8 μg/mL kanamycin and 0.45 μg/mL polymyxin B; with C for the combination antibiotic containing 20 μg/mL kanamycin and 1 μg/mL polymyxin E, whereas the control group was subjected to monotherapy with either 20 μg/mL kanamycin or 1 μg/mL polymyxin E; with D for the combination drugs containing (1) 30 μg/mL ampicillin and 0.9 μg/mL polymyxin B, and (2) 0.15 μg/mL ciprofloxacin and 0.9 μg/mL polymyxin B, respectively, whereas the control group was treated with monotherapy using 30 μg/mL ampicillin, 0.15 μg/mL ciprofloxacin, or 0.9 μg/mL polymyxin B, respectively.

FIG. 7 shows the highly effective eradication of metG2 high-persister bacteria and wild-type E. coli persisters by the polymyxin B-kanamycin combination drug, with A for the E. coli metG2 strain, and B for the wild-type strain. Both were treated with the combination antibiotic containing 3 MIC polymyxin B and 2.5 MIC kanamycin, whereas the the control group was subjected to monotherapy with either 2.5 MIC kanamycin or 3 MIC polymyxin B.

FIG. 8 shows the highly effective eradication of exponential-phase wild-type E. coli persisters by the polymyxin B-kanamycin combination drug. The experimental group was treated with the combination antibiotic containing 3 MIC polymyxin B and 2.5 MIC kanamycin, while the control group was treated monotherapy.

FIG. 9 shows the highly effective eradication of hipA7 high-persister bacteria by various combination antibiotics of polymyxin B and aminoglycosides, with A for the combinations containing 0.9 μg/mL polymyxin B with either 16 μg/mL amikacin or 16 μg/mL streptomycin; with B for the combination of 0.9 μg/mL polymyxin B and 16 μg/mL tobramycin; with C for the combination of 0.9 μg/mL polymyxin B and 12 μg/mL gentamicin; with D for a uniform concentration of 6 μg/mL for all aminoglycoside molecules in the combinations.

FIG. 10 shows the highly effective eradication of metG2 high-persister bacteria by various combination antibiotics of polymyxin B and aminoglycosides, with A for the combination containing 0.9 μg/mL polymyxin B and 6 μg/mL amikacin; with B for the combination containing 0.9 μg/mL polymyxin B and 6 μg/mL streptomycin; with C for the combination containing 0.9 μg/mL polymyxin B and 6 μg/mL tobramycin; and with D for the combination containing 0.9 μg/mL polymyxin B and 6 μg/mL gentamicin, while the control group was treated monotherapy.

FIG. 11 shows the highly effective eradication of wild-type E. coli persisters by various combination antibiotics of polymyxin B and aminoglycosides, with A for the combination containing 0.9 μg/mL polymyxin B and 6 μg/mL amikacin; with B for the combination containing 0.9 μg/mL polymyxin B and 6 μg/mL streptomycin; with C for the combination containing 0.9 μg/mL polymyxin B and 6 μg/mL tobramycin; with D for the combination containing 0.9 μg/mL polymyxin B and 12 μg/mL gentamicin, while the control group was treated monotherapy.

FIG. 12 shows the bactericidal effects of polymyxin-aminoglycoside combination antibiotics on clinical pathogenic bacteria, with A for Klebsiella pneumoniae clinical isolate 9030; with B for Klebsiella pneumoniae clinical isolate 9182; with C for Escherichia coli clinical isolate 0005; with D for Escherichia coli clinical isolate 0011; with E for Pseudomonas aeruginosa clinical isolate 1128; and with F for Staphylococcus aureus clinical isolate 1115.

FIG. 13 shows the loss of hipA7 cell membrane potential caused by the combination of polymyxin B and kanamycin, with A for the combination antibiotic containing 0.9 μg/mL polymyxin B and 20μg/mL kanamycin, with B for 20 μg/mL kanamycin alone, and C for 0.9 μg/mL polymyxin B.

FIG. 14 shows the disruption of hipA7 cell membranes by the polymyxin B-kanamycin combination antibiotic, with A for the 2-hour time and with B for the 4-hour time.

FIG. 15 shows the disruption of hipA7 cell membranes by the polymyxin B-kanamycin combination antibiotic, with A for the combination of 0.9 μg/mL polymyxin B and 20 μg/mL kanamycin; with B for 20 μg/mL kanamycin alone; and with C for 0.9 μg/mL polymyxin B alone.

FIG. 16 shows the safety evaluation of polymyxin-aminoglycoside combination antibiotics to human cells.

### EMBODIMENTS OF THE INVENTION

The present invention discloses for the first time the use of a combination drug comprising a polymyxin antibiotic and an aminoglycoside antibiotic for inhibiting and eradicating pathogenic bacteria, particularly in the preparation of a medicament for treating persistent bacterial infections. Specifically, the pathogenic bacteria are “nutrient-insensitive” persisters. Herein, the polymyxin antibiotic comprises polymyxin or the salt, hydrate, or solvate thereof; and the aminoglycoside antibiotic comprises an aminoglycoside or the salt, hydrate, or solvate thereof.

In the present invention, the active ingredients of the drug are polymyxin antibiotics and aminoglycoside antibiotics, further comprising a carrier.

Given that polymyxins represent a class of molecules with similar structures and functions, the polymyxin components in the combination drugs disclosed herein are not limited to polymyxin B and polymyxin E, but also include other molecules with similar structures and functions. The content ratio of polymyxin antibiotics in finished polymyxin-aminoglycoside compound antibiotics ranges from 0.001% to 95%, enabling a bactericidal concentration range of 0.001 to 200 μg/mL during the bactericidal process.

Given that aminoglycoside antibiotics constitute a class of molecules with analogous structures and functions, the scope of the combination drug disclosed in the present invention regarding the aminoglycoside antibiotic is not limited to clinically common agents such as kanamycin, amikacin, tobramycin, gentamicin, streptomycin, and netilmicin, but also includes other aminoglycoside drug molecules with similar structures and functions. The content of the aminoglycoside antibiotic in the finished combination drug ranges from 0.01% to 99.5% by weight, thereby achieving an applicable concentration range of 0.01 to 200 μg/mL during the bactericidal process.

The above two ratios (content ratios) are calculated based on the total weight of “polyoxytetracycline antibiotics and aminoglycoside antibiotics” as 100%, excluding other components such as carriers. If other carrier components are included, the calculation is proportionally adjusted according to the weight.

As common sense, the term “pharmaceutically acceptable salts” refers to modified compounds or derivatives through preparation of their acid or base salts. Examples of pharmaceutically acceptable salts include: inorganic acid salts or organic acid salts of basic groups such as amines, alkali metal salts or organic salts of acidic groups like carboxylic acids, conventional non-toxic salts or quaternary ammonium salts. Conventional non-toxic salts include inorganic acids such as hydrochloric acid, hydrobromic acid, sulfuric acid, aminesulfonic acid, phosphoric acid, and nitric acid; or organic acids such as acetic acid, propionic acid, succinic acid, glycolic acid, stearic acid, lactic acid, malic acid, tartaric acid, citric acid, ascorbic acid, diphenylhydantoin, maleic acid, hydroxymaleic acid, phenylacetic acid, glutamic acid, benzoic acid, salicylic acid, sulfamic acid, 2-acetoxybenzoic acid, fumaric acid, tosylic acid, methanesulfonic acid, ethanedisulfonic acid, oxalic acid, and hydroxyethyl sulfonic acid. Pharmaceutically acceptable salts can be synthesized through conventional chemical methods from parent compounds containing basic or acidic components. In some cases, such salts can be prepared by reacting the free acid or base form of these compounds with stoichiometric amounts of appropriate base or acid in water, organic solvents, or their mixtures; preferred organic solvents include ethyl ether, ethyl acetate, ethanol, isopropanol, or acetonitrile. As common sense, active ingredients include all pharmaceutically acceptable salts, hydrates, and/or solvates of compounds. Certain functional groups such as hydroxyl, amino, etc., form complexes and/or coordination compounds with water and/or various solvents in various physical forms of the compounds.

As common sense, the carrier in pharmaceutical terminology refers to a pharmaceutically acceptable substance recognized in the field. This includes materials, compositions, or media such as liquid or solid fillers, diluents, excipients, solvents, or encapsulating materials that facilitate the transport of active pharmaceutical ingredients. Common examples of pharmaceutically acceptable carriers include: sugars (e.g., lactose, glucose, sucrose); starches (e.g., corn starch, potato starch); cellulose derivatives (e.g., sodium carboxymethyl cellulose, ethyl cellulose, cellulose acetate); powdered tragacanth; malt; gelatin; talc; excipients (e.g., cocoa butter, suppository wax); oils (e.g., peanut oil, cottonseed oil, safflower oil, sesame oil, olive oil, corn oil, soybean oil); diols (e.g., propylene glycol); polyols (e.g., glycerol, sorbitol, mannitol, polyethylene glycol); esters (e.g., ethyl oleate, ethyl laurate); agar; buffers (e.g., magnesium hydroxide, aluminum hydroxide); alginic acid; water; isotonic saline; Ringer's solution; ethanol; phosphate buffer solutions; and other non-toxic compatible substances used in pharmaceutical formulations.

The administration methods for the combination drugs of this invention are unrestricted and include oral, intravenous, intramuscular, subcutaneous, transdermal, inhalation, oral, ocular, sublingual, vaginal, and rectal routes. The combination drugs described herein can be administered in unit dosage forms and/or formulations containing conventional non-toxic pharmaceutically acceptable carriers, adjuvants, and carriers. Exemplary oral administration forms include tablets, capsules, elixirs, syrups, etc. Parenteral administration routes include intravenous, intrarortorial, intraperitoneal, epidural, introurethral, intrasternal, intramuscular, and subcutaneous routes, as well as any other parenteral administration methods recognized in the field. Administration methods include needle (including microneedle) syringes, needle-free syringes, infusion techniques, and any other parenteral administration methods recognized in the field. Parenteral preparations are typically aqueous solutions containing carriers such as salts, carbohydrates, and buffers, and can also be formulated as sterile non-aqueous solutions or lyophilized forms for use with appropriate carriers (e.g., sterile pyrogen-free water). The preparation of parenteral preparations under sterile conditions, such as through lyophilization, can be easily accomplished using standard pharmaceutical techniques familiar to those skilled in the art.

The dosage of the combination drug in this invention is conventional and can be selected based on administration methods, the condition to be treated, severity of the condition, whether it is for treatment or prevention, as well as the patient's age, weight, and health status. In this invention, polymyxin and aminoglycoside antibiotics can be administered simultaneously through any suitable method, differing from conventional sequential administration, separate administration, or administration in a individual drug formulation.

The specific drug used in the present invention is the existing product, the specific preparation and experimental method is the conventional technology, the test is the existing laboratory technology, the test strain is mainly the commercial standard strain, and the clinical isolates used are the infectious bacterial isolates available in any hospital.

The proportion of persisting cells in bacterial cultures typically reaches higher levels during the nutrient-depleted stationary phase. To investigate the persistence capacity of the model bacterium Escherichia coli(E. coli) against most bactericidal antibiotics, the standard BW25113 strain with E. coli K12 background was cultured at 37° C. in commercially available LB liquid medium until reaching the stationary phase. The culture was then inoculated at a 1:20 dilution into fresh LB liquid medium containing antibiotics at multiple dilutions of the minimum inhibitory concentration (MIC). Samples were collected after different treatment durations, and residual antibiotics were removed through two centrifugations. After tenfold serial dilution in physiological saline, the cultures were plated on LB agar plates without antibiotics. Survival colony-forming units (CFUs) were counted after two days of incubation, with initial CFU values before antibiotic addition serving as controls to calculate bacterial survival rates and persistence levels using conventional experimental methods. Testing for other bacteria followed the same protocol. The antibiotics used included classical bactericidal agents: β-lactams (inhibiting cell wall synthesis, e.g., ampicillin), fluoroquinolones (causing double-stranded DNA breaks, e.g., ciprofloxacin), aminoglycosides (acting on ribosomes to induce translation errors, e.g., kanamycin), polymyxins (interfering with cell membrane function, e.g., polymyxin B), and mitomycin (causing double-stranded DNA breaks through GC bridge formation, e.g., mitomycin C). The drug dosage employed a concentration capable of killing sensitive bacteria, typically a publicly known multiple of the MIC, and the specific concentration can be calculated based on the MIC and the multiple.

Fluorescence microscopy and flow cytometry are used to detect intracellular fluorescent substances in bacteria according to the standard procedures and detection methods of the instruments or reagents.

The above describes the general methodologies employed in the bacterial experiments of the present invention, which also represent common analytical approaches documented in the literature for assessing bacterial persistence levels. All antibiotics used in this invention are routinely applied in clinical practice. The experimental results demonstrate that conventional monotherapies with existing antibiotics show poor bactericidal efficacy against persister cells, and no reports have been found regarding the use of combination drugs to treat bacterial persistence.

1. Antibiotics at high concentrations and for long periods of time cannot effectively kill the “nutrient-insensitive” persistent bacteria in the wild-type E. coli population.

In the stationary phase population of wild-type Escherichia coli, a small fraction of cells exhibits high persistence. When diluted into nutrient-rich medium for killing, these persistent cells can survive even after exposure to high-concentration bactericidal antibiotics, demonstrating resistance to clinically common antibiotics, which are defined as “nutrient-insensitive” persistent bacteria.

When wild-type E. coli BW 25113 cultures in the stationary phase were exposed to kanamycin (24 μg/mL, 3 MIC) exceeding the peak serum concentration (20 μg/mL) for 10 hours, their viability decreased by 100,000-fold, with the persistence survival level maintained at 0.001%. Similarly, cultures treated with ciprofloxacin (0.6 μg/mL, 20 MIC), ampicillin (120 μg/mL, 20 MIC), or polymyxin B (0.9 μg/mL, 3 MIC) for 10 hours also maintained a persistence rate of 0.001% (see FIG. 1). These results indicate that approximately 1 in 100,000 cells in stationary-phase E. coli cultures exhibit a “nutrient-insensitive persistence” state. These cells, in the presence of nutrients, survive exposure to high concentrations of various clinically common bactericidal antibiotics for extended duration through persistence, and resume growth and proliferation upon antibiotic removal.

In the stationary-phase cultures of Escherichia coli, the vast majority of cells exhibit a “nutrient-sensitive” persistence/tolerance state. Unlike the small fraction of “nutrient-insensitive” persisters mentioned above, most cells in E. coli BW 25113 stationary-phase cultures show minimal viability when exposed to conditions lacking key nutrients or completely deprived of exogenous nutrients, which prevents antibiotics from reaching their targets or generating sufficient reactive oxygen species (ROS), resulting in negligible cell death. However, when diluted into nutrient-rich medium and treated with antibiotics, these stationary-phase cultures can be rapidly and extensively eliminated by various clinically common bactericidal antibiotics (see FIG. 1). These cells, which survive under nutrient starvation but are killed by external nutrients, were previously misclassified as persisters. Their survival phenotype should actually be categorized as tolerance rather than true persistence. Therefore, the term “persistence” in this invention specifically refers to “nutrient-insensitive” persistence.

FIG. 1 shows the survival capacity of wild-type E. coli after exposure to different bactericidal antibiotics. In A, a stationary-phase culture of wild-type E. coli BW25113 was inoculated into fresh LB liquid medium at a 1:20 ratio, immediately followed by the addition of kanamycin at 3 MIC (MIC=8 μg/mL). The culture was then incubated at 37° C. with shaking at 160 rpm for 10 hours. Samples were collected at various time points, washed to remove the drug, serially diluted ten-fold in physiological saline, spotted onto LB agar plates, and incubated for 2 days before determining the number of viable cells (CFU); the conditions for B, C, and D are the same as for A, except that the antibiotics used for eradication were 20 MIC ciprofloxacin (MIC=0.03 μg/mL), 20 MIC ampicillin (MIC=6 μg/mL), or 3 MIC polymyxin B (MIC=0.3 μg/mL), respectively. Each measurement was obtained from at least three independent experiments, with error bars representing the mean±standard deviation.

2. The E. coli hipA7 and metG2 strains exhibit high-level persistence, with approximately 10% of cells in their stationary-phase cultures capable of surviving eradication by any individual drug from the five major classes of bactericidal antibiotics.

E. coli hipA7 and metG2, as two representative high-persistence strains, can be obtained through commercial purchase or constructed according to literature methods for further evaluation of antibiotic efficacy against high-persistence cells. The hipA7 strain, obtained in 1983 through evolutionary screening, is the first recognized high-persistence strain and is widely used in studies of persistence mechanisms. The methionyl-tRNA synthetase gene metG2 mutant strain, reported in 2012 and obtained via transposon library screening, exhibits a high persistence level comparable to that of the hipA7 strain.

Approximately 10% of cells in the stationary phase culture of the hipA7 strain exhibited strong persistence to five types of bactericidal antibiotics, demonstrating survival against any individual antibiotic treatment. When cultured in fresh LB medium containing 3 MIC kanamycin for 10 hours, the hipA7 strain maintained a survival rate of 10%, which was 10,000 times higher than that of the wild-type E. coli. Similarly, after exposure to 20 MIC ciprofloxacin, 20 MIC ampicillin, 3 MIC polymyxin B, or 3 MIC mitomycin C, the hipA7 culture maintained approximately 10% viability (see FIG. 2). FIG. 2 demonstrates the high survival capacity of the hipA7 strain against various bactericidal antibiotics. In A, the stationary-phase cultures of E. coli hipA7 and wild-type strains were diluted 1:20 into fresh LB liquid medium, immediately followed by the addition of kanamycin at 3 MIC, and incubated at 37° C. with shaking at 160 rpm for 10 h and the viable cell counts were determined at specified time points under the same conditions as described for A in FIG. 1. The experimental conditions were identical to those of A in FIG. 1, except that the antibiotics used for eradication were 3 MIC streptomycin, 20 MIC ciprofloxacin, 20 MIC ampicillin, 3 MIC polymyxin B, or 3 MIC mitomycin C (MIC=0.8 μg/mL), respectively. Each measurement was obtained from at least three independent experiments, with error bars representing the mean±standard deviation.

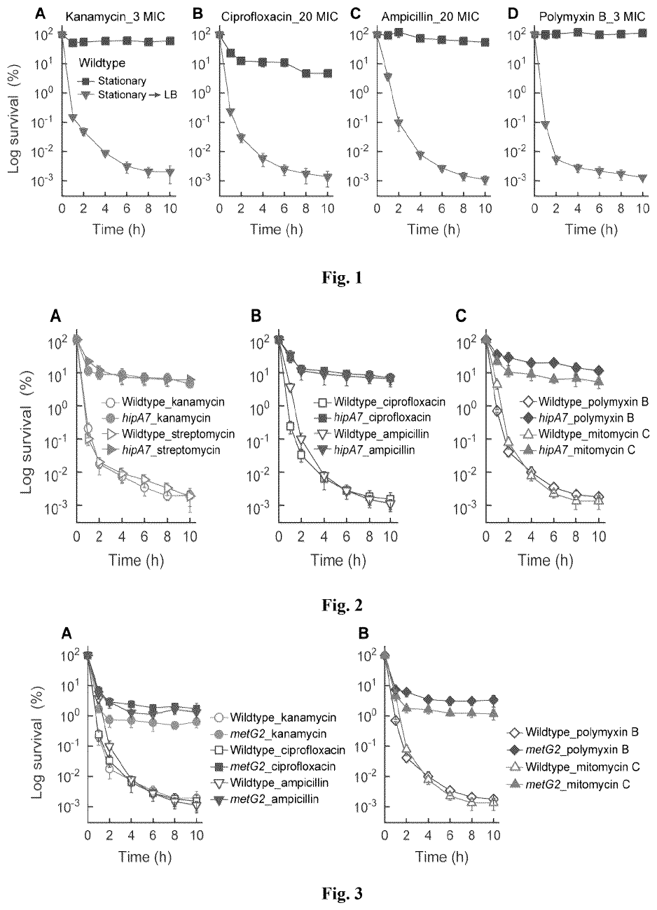

In the stationary phase cultures of metG 2 strain, approximately 1-10% of cells maintained strong persistence to five bactericidal antibiotics. Similar to hipA7, after 10-hour exposure to individual antibiotics including 20×MIC ciprofloxacin, 20×MIC ampicillin, 3×MIC streptomycin, 3×MIC polymyxin B, or 3×MIC mitomycin C, the survival rate remained at 1-10% (FIG. 3). FIG. 3 demonstrates metG2's high persistence to various bactericidal antibiotics. Specifically, stationary phase cultures of E. coli metG2 and wild-type strains (A and B) were inoculated at a 1:20 ratio into fresh LB liquid medium, followed by the addition of 3 MIC kanamycin, 20 MIC ciprofloxacin, 20 MIC ampicillin, 3 MIC polymyxin B, or 3 MIC mitomycin C, respectively. Survival cell counts were measured at different time points under conditions identical to those shown in FIG. 1(A). Each measurement was obtained from at least three independent experiments, with error bars representing the mean±standard deviation.

The results demonstrate that the ‘nutrient-insensitive’ persistent bacterial population in the stationary phase cultures of E. coli hipA 7 and metG 2 strains increased by tens of thousands of times compared to the wild-type strain. Notably, even under conditions of abundant exogenous nutrients, no clinically commonly used bactericidal antibiotics (including aminoglycosides and polymyxins) could effectively eliminate these persistent bacterial cells when administered as monotherapy.

3. The sequential combination of bactericidal antibiotics failed to effectively eliminate both wild-type E. coli and persistent cells of the hipA7 and metG2 strains.

To evaluate the combined antimicrobial effects of sequential antibiotic treatments on residual bacterial viability, stationary-phase cultures of wild-type E. coli and hipA7/metG2 strains were cultured under sufficient exogenous nutrients. After 5 hours of treatment with one antibiotic, the antibiotic was washed off, and the cultures were switched to another antibiotic targeting a different mechanism for 5 consecutive hours. The survival rates were then measured.

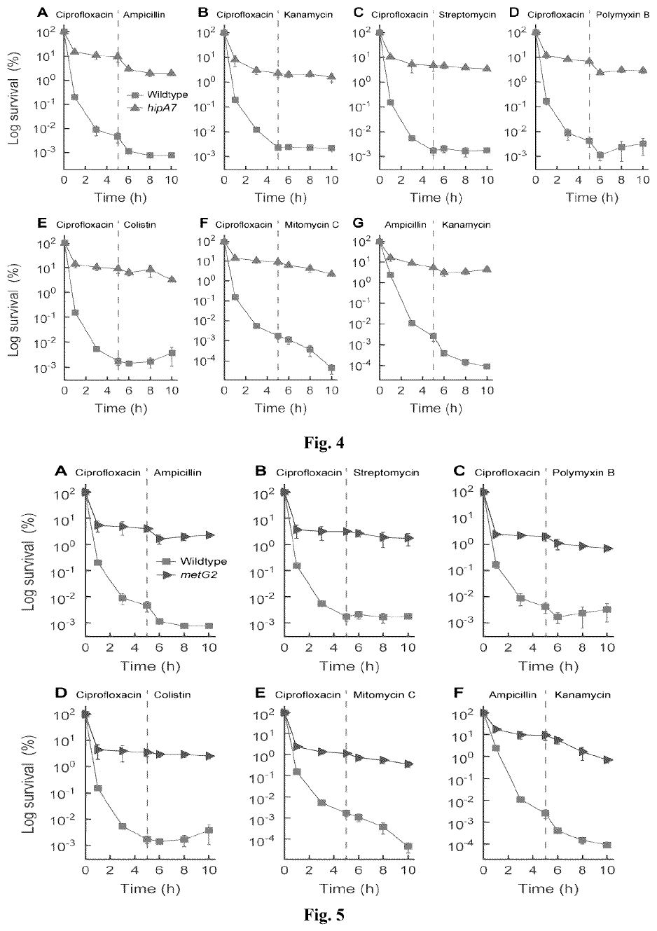

When hipA7 bacterial cultures were treated with 20 MIC ciprofloxacin for 5 hours, they were immediately switched to another target-type antibiotic (such as 20 MIC ampicillin, 3 MIC kanamycin, 3 MIC streptomycin, 3 MIC polymyxin B/E [Colistin], or 3 MIC mitomycin C) for continued treatment, maintaining a cell survival rate of 3-10%. Similarly, hipA7 cultures treated with 20 MIC ampicillin for 5 hours followed by 3 MIC kanamycin for 5 hours also maintained a survival rate of 3-10% (see FIG. 4). However, when treated with the same antibiotic combinations, wild-type E. coli cultures showed a remarkably low survival rate of 0.001-0.0001%. FIG. 4 demonstrates the high survival viability of the hipA7 strain under combined antibiotic treatments, with the wild-type strain serving as control. In A, E. coli hipA7 and wild-type cultures were diluted 1:20 into fresh LB liquid medium, immediately added with 20 MIC ciprofloxacin, and incubated at 37° C. with 160 rpm shaking for 5 h. Cells were collected by centrifugation, washed twice to remove the drug, resuspended in fresh LB medium containing 20 MIC ampicillin, and cultured for an additional 5 h. The surviving cell count under condition A (FIG. 1) was measured. Conditions of B-G were identical to that of A, with different antibiotic combinations: 20 MIC ciprofloxacin, 3 MIC kanamycin, 3 MIC streptomycin, 3 MIC polymyxin B/E, 3 MIC mitomycin C, and 20 MIC ampicillin, respectively. Each measurement was obtained from at least three independent experiments, with error bars representing the mean±standard deviation.

The metG2 bacterial strain cultures exhibited high persistence viability after sequential combination treatments with different antibiotics. For example, after 5 hours of eradication with 20 MIC ciprofloxacin, and switching to 20 MIC ampicillin, 3 MIC streptomycin, or 3 MIC polymyxin B for an additional 5 hours of treatment resulted in a persistent survival level of 1-5% (FIG. 5). FIG. 5 demonstrates metG2's high persistence viability under different antibiotic combinations, with the wild-type strain as control; In A, E. coli metG2 and wild-type cultures were diluted 1:20 to fresh LB liquid medium, immediately added with 20 MIC ciprofloxacin, and incubated at 37° C. with 160 rpm shaking for 5 hours. Cells were collected by centrifugation, washed twice to remove antibiotics, resuspended in fresh LB medium containing 20 MIC ampicillin, and cultured for 5 hours. The survival cell count under condition A in FIG. 1 was measured. Conditions of B-F were identical to that of A, but with different antibiotic combinations: 20 MIC ciprofloxacin, 3 MIC kanamycin, 3 MIC streptomycin, 3 MIC polymyxin B/E, 3 MIC mitomycin C, and 20 MIC ampicillin, respectively. Each measurement was obtained from at least three independent experiments, with error bars representing the mean±standard deviation.

The aforementioned results indicate that the persistent phenomenon characterized by broad-spectrum resistance to all bactericidal antibiotics (particularly the nutrient-insensitive type) is present in cultures of various bacterial strains, such as wild-type E. coli and its mutant strains hipA7 and metG2. These pan-antibiotic persistent bacterial cells likely possess resistance mechanisms analogous to those of spores or cysts, conferring exceptional resistance to nearly all classes of bactericidal antibiotics currently available on the market. They can survive prolonged exposure to high antibiotic concentrations and remain viable even when subjected to alternating combinations targeting different antimicrobial targets. This persistent survival mechanism continues to pose an unresolved clinical challenge in anti-infective therapy.

### EMBODIMENT 1

The combination drug of polymyxin and aminoglycoside antibiotics can effectively eradicate various persisting bacteria in the clinical serum concentration range.

Under experimental conditions at or below clinical serum concentrations, various antibiotic combinations formed by polymyxin with individual aminoglycoside molecules all demonstrated rapid and potent eradication of all tested persister bacteria, achieving the goal of swift and substantial elimination of persisters within a short timeframe. It is known that the peak serum concentration of kanamycin is approximately 18-20 μg/mL, which corresponds to about 2.5 MIC for E. coli BW25113; the clinical dosage guideline for polymyxin is 2 mg/kg, yielding an average serum concentration of approximately 2.8 μg/mL (range: 0.68-4.88 μg/mL).

In the experiment, a compound antibiotic composed of polymyxin and aminoglycoside was used. The two classes of antibiotics were pre-mixed to form a combination drug with different ratios before application, thereby optimizing the synergistic effect of the two drug components. For in-vitro experiments, the two drugs can also be added simultaneously to the same culture at a specific ratio for bactericidal purposes. In the embodiments, the unit “mL” in the drug concentrations refers to the volume of the culture medium.

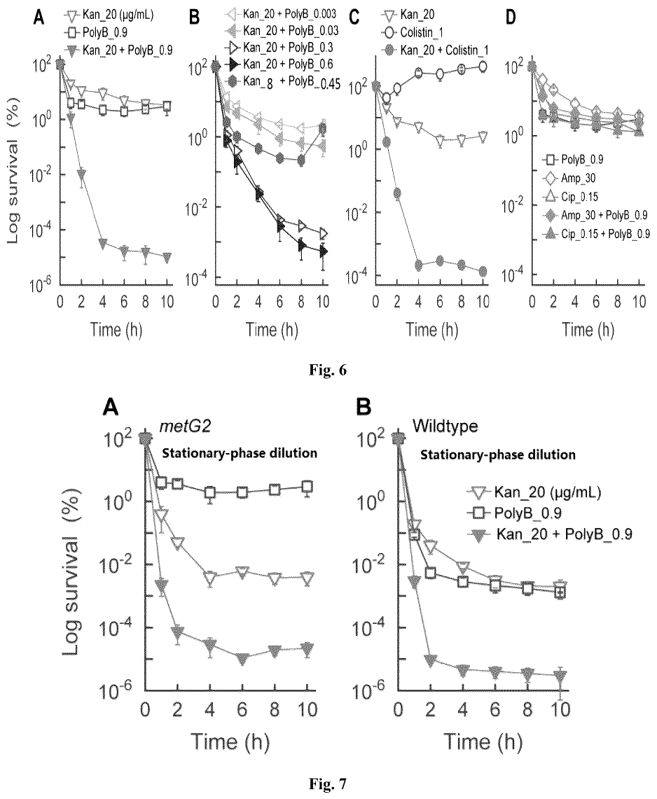

As shown in FIG. 6, the combination drug of polymyxin B and kanamycin effectively eliminates hipA7 high-persistence bacteria. In A, a stationary-phase culture of E. coli hipA7 strain was diluted 1:20 into fresh LB medium, followed by immediate addition of a compound drug consisting of kanamycin (final concentration 20 μg/mL) and polymyxin B (0.9 μg/mL). The control groups were treated with either 20 μg/mL kanamycin or 0.9 μg/mL polymyxin B as monotherapy. The cultures were incubated at 37° C. with shaking at 160 rpm for 10 hours, and the count of surviving cells at different time points was determined under the conditions specified in FIG. 1A; the conditions for B were identical to that of A, except that the combination drug containing kanamycin of 20 μg/mL and polymyxin B of 0.003, 0.03, 0.3, or 0.6 μg/mL, respectively; alternatively, a combination comprising 8 μg/mL kanamycin and 0.45 μg/mL polymyxin B; the conditions for C were identical to that of A, except that the combination antibiotic containing 20 μg/mL kanamycin and 1 μg/mL polymyxin E (also known as Kristin), whereas the control group was subjected to monotherapy with either 20 μg/mL kanamycin or 1 μg/mL polymyxin E; the conditions for D were identical to that of A, except that the combination drugs containing (1) 30 μg/mL ampicillin and 0.9 μg/mL polymyxin B, and (2) 0.15 μg/mL ciprofloxacin and 0.9 μg/mL polymyxin B, respectively, whereas the control group was treated with monotherapy using 30 μg/mL ampicillin, 0.15 μg/mL ciprofloxacin, or 0.9 μg/mL polymyxin B, respectively. Each measurement was obtained from at least three independent experiments, with error bars representing the mean±standard deviation. The stationary-phase culture of the hipA7 strain was diluted into fresh LB medium and immediately subjected to killing by the addition of a compound drug consisting of polymyxin B and kanamycin at concentrations of 0.9 μg/mL (3 MIC) and 20 μg/mL (2.5 MIC), respectively; the survival rate of hipA 7 persisters rapidly decreased by 7 orders of magnitude, with persistence levels as low as 10⁻⁷ to 10⁻⁸. Compared to the control groups treated with either 2.5 MIC kanamycin or 3 MIC polymyxin B alone (which exhibited persistence levels of 5-10%), this represents a reduction of at least one million-fold. When 2.5 MIC kanamycin was combined with lower concentrations of polymyxin B (0.01, 0.1, 1, and 2 MIC), the persister survival level of hipA7 decreased by 10 to 10,000-fold after drug treatment. Similarly, when the concentrations of polymyxin B and kanamycin in the combination were reduced to 1.5 MIC (0.45 μg/mL) and 1 MIC (8 μg/mL), respectively, the persistence level of hipA7 dropped by more than 10-fold. Therefore, the combination of polymyxin B and kanamycin can rapidly and substantially kill highly persistent hipA7 bacteria across a wide range of concentrations achievable at clinical serum levels. Similarly, the combination of polymyxin E and kanamycin also rapidly eliminates persisters. When 2.5 MIC kanamycin was combined with 1.0 μg/mL (10 MIC) polymyxin E, the persistence level of hipA7 was reduced by at least 10,000-fold. In contrast, combinations of polymyxin B with either ampicillin or ciprofloxacin showed no significant change in the persistence level of hipA7 compared to the control group by monotherapy treatment, remaining around 5-10%. This result indicates that not all drug combinations or arbitrary combinations lead to better technical outcomes compared to individual drugs. In particular, the combination of polymyxins with cell wall-disrupting β-lactams or DNA-cleaving quinolone antibiotics exhibits no synergistic effect in eradicating persisters. This also indicates that the potential for synergistic interactions between polymyxins and aminoglycoside combination drugs to rapidly eliminate persistent bacteria remains unpredictable.

To demonstrate the synergistic bactericidal effect between polymyxins and aminoglycosides, the conventional method was used to calculate their FICI (i.e., the fractional inhibitory concentration index, where a value ≤0.5 indicates a significant synergistic effect). The FICI value for the combination of kanamycin and polymyxin B was 0.25, while that for the combination with polymyxin E was 0.4. Therefore, the formulation of a combination drug comprising a polymyxin and an aminoglycoside antibiotic not only rapidly kills persisters but also reduces the concentration required for each drug to inhibit bacterial growth through synergistic effects.

### EMBODIMENT 2

A combination comprising polymyxin and aminoglycosides is effective to rapidly and substantially kill bacterial persisters, including both metG2 high-persistence bacteria and wild-type E. coli low-persistence bacteria.

FIG. 7 shows the highly effective eradication of metG2 high-persister bacteria and wild-type E. coli persisters by the polymyxin B-kanamycin combination drug, with A for the E. coli metG2 strain, and B for the wild-type strain. The stationary-phase cultures of these strains were diluted 1:20 into fresh LB medium, followed by immediate addition of the combination antibiotic containing 3 MIC of polymyxin B and 2.5 MIC of kanamycin. The control group was subjected to monotherapy treatment with 2.5 MIC kanamycin and 3 MIC polymyxin B. Cell survival rates at different killing stages were measured under conditions A in FIG. 1. After treatment with the polymyxin B-kanamycin combination, the survival rate of metG2 strains decreased by seven orders of magnitude, with persistence levels dropping by 10² to 10⁶-fold. Similarly, wild-type E. coli exhibited nearly a thousandfold reduction in persistence levels after exposure to the combination antibiotic, decreasing from 0.001% to 0.000001% compared to monotherapy treatment. These results demonstrate that the combination of polymyxin and aminoglycoside antibiotics can efficiently eliminate E. coli persistence strains induced by various factors and at different survival levels.

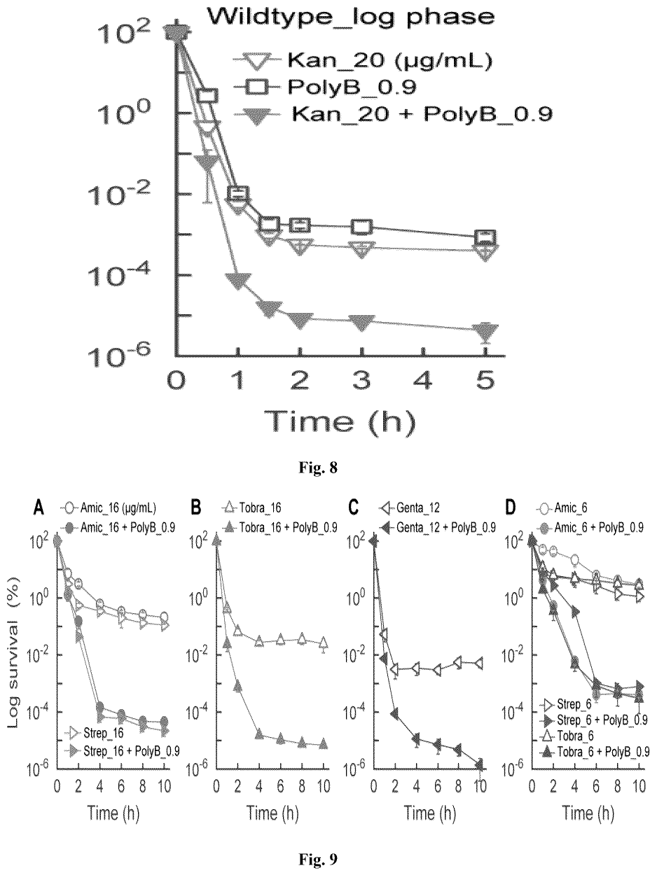

Polymyxin-Aminoglycoside combination antibiotics not only effectively eliminate persistent bacteria in stationary phase cultures but also efficiently kill sensitive cells during exponential growth and “nutrient-insensitive” persistent bacteria, reducing their survival levels by seven orders of magnitude and decreasing persistence by 100-fold (see FIG. 8). This demonstrates the potent bactericidal effect of the polymyxin B-kanamycin combination on wild-type E. coli persistence. Fresh LB liquid medium was inoculated with 1:200 dilution of stationary-phase cultures of wild-type E. coli, which were incubated under shaking until reaching the exponential phase (OD600=0.3). The cultures were immediately treated with either the combination antibiotic containing 3 MIC of polymyxin B and 2.5 MIC of kanamycin, or administered as a individual agent, with cell survival rates measured at various time points. Each measurement was obtained from at least three independent experiments, with error bars representing the mean±standard deviation. The results indicate that the polymyxin-aminoglycoside combination effectively eliminates bacteria regardless of whether they are in the metabolically active exponential growth phase or in a nutrient-insensitive persistent state. Among wild-type cells, approximately 0.001% were identified as nutrient-insensitive persistent bacteria, which were also successfully eradicated by the combination antibiotic, reducing persistence levels by approximately 100-fold.

### EMBODIMENT 3

The combination of polymyxin with common aminoglycoside molecules has similar high efficiency in killing and clearing the persistence bacteria.

Upon confirming the synergistic bactericidal effects of the kanamycin-polymyxin combination, the efficacy of other aminoglycoside molecules in combination with polymyxin against bacterial persisters was further evaluated, including streptomycin, amikacin, tobramycin, and gentamicin. Although the peak blood concentrations of most of these aminoglycosides can exceed 16 μg/mL, the concentrations used in this test did not exceed 16 μg/mL, with polymyxin B maintained at 0.9 μg/mL. Theoretically, higher concentrations of these antibiotics would be expected to enhance bactericidal effectiveness.

As shown in FIG. 9, the combination of polymyxin B and aminoglycosides effectively eliminated hipA7 high-persistence strains. E. coli hipA7 cultures in the stationary phase were diluted 1:20 into fresh LB medium and immediately treated with antibiotics, with A for the combinations containing 0.9 μg/mL polymyxin B with either 16 μg/mL amikacin or 16 μg/mL streptomycin; with B for the combination of 0.9 μg/mL polymyxin B and 16 μg/mL tobramycin; with C for the combination of 0.9 μg/mL polymyxin B and 12 μg/mL gentamicin; with D for a uniform concentration of 6 μg/mL for all aminoglycoside molecules in the combinations, while the control group was treated by monotherapy. Cell survival rates were measured at different time points under conditions A in FIG. 1. Each measurement was obtained from at least three independent experiments, with error bars representing the mean±standard deviation. After treatment with polymyxin B combined with 16 μg/mL streptomycin, amikacin, tobramycin, or 12 μg/mL gentamicin, the viability of hipA 7 cultures in the stationary phase decreased by 10⁶-10⁷ fold, with persistence survival levels dropping to 0001-0.00001%. By reducing the concentration of aminoglycosides in the combination antibiotics to 6 μg/mL, the persistence viability of hipA7 stationary cultures was reduced to 0.001-0.0001%, which was only about one order of magnitude higher than the survival level obtained by the combination drug containing 16 μg/mL aminoglycosides.

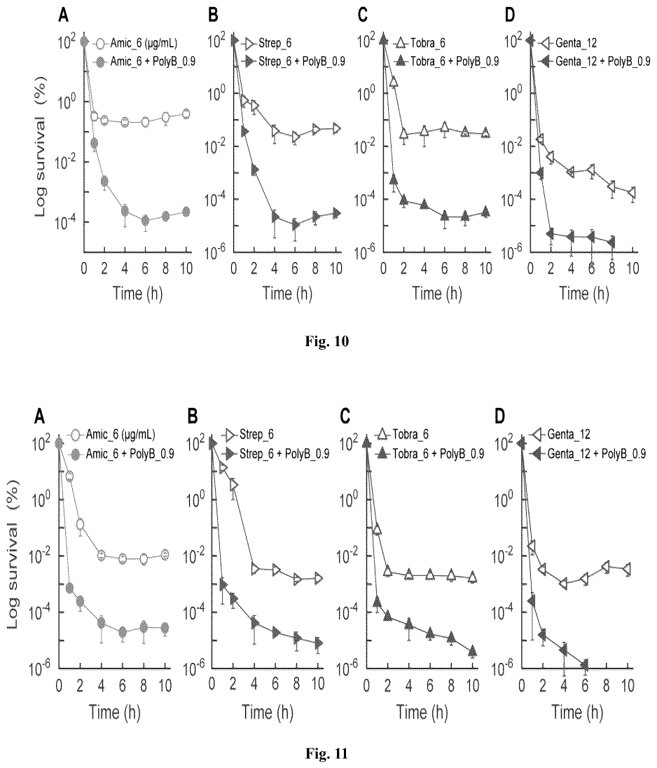

Referring to FIG. 10, it illustrates the highly effective killing of metG2 high-persistence bacteria by various combinations of polymyxin B and aminoglycosides. A to D correspond to experiments with the E. coli metG2 strain. The conditions for bacterial culture, combination antibiotic concentrations, treatment procedures, and survival assays were all consistent with those described in FIG. 9C and FIG. 9D. Each measurement was obtained from at least three independent experiments, with error bars representing the mean±standard deviation. After treatment with combinations of polymyxin and 6 μg/mL of the aforementioned aminoglycosides, the persister survival level of metG2 stationary-phase cultures was reduced to 0.0001%-0.00001%. Similarly, treatment with the combination antibiotics also reduced the persister survival level of wild-type E. coli to 0.0001%-0.00001%. As shown in FIG. 11, it illustrates the highly effective killing of wild-type E. coli persisters by various combinations of polymyxin B and aminoglycosides. A to D correspond to experiments with the wild-type E. coli BW25113 strain. The conditions for bacterial culture, combination antibiotic concentrations, treatment procedures, and survival assays were all based on those described in FIG. 9C and FIG. 9D. Each measurement was obtained from at least three independent experiments, with error bars representing the mean±standard deviation. In summary, all combination drugs comprising clinically common aminoglycoside molecules and polymyxin exhibit potent killing and eradication against bacterial persisters.

### EMBODIMENT 4

Polymyxin-aminoglycoside combination antibiotics have a broad-spectrum bactericidal efficacy against persister bacteria in both Gram-negative and Gram-positive bacteria.

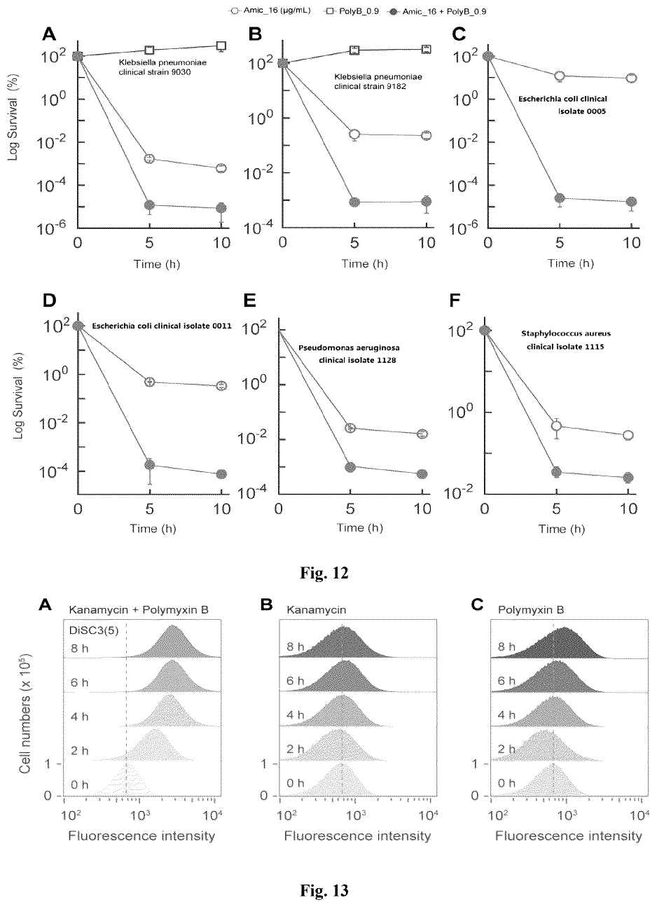

This invention demonstrates that the combination antibiotics have a broad-spectrum bactericidal efficacy against persister bacteria in both Gram-negative and Gram-positive bacteria. Several typical clinically relevant pathogenic bacteria were selected for testing, including Gram-negative clinical strains of Escherichia coli, Klebsiella pneumoniae, and Pseudomonas aeruginosa, and a Gram-positive clinical strain of Staphylococcus aureus.A representative combination drug comprised 0.9 μg/mL polymyxin B and 16 μg/mL amikacin. FIG. 12 shows the bactericidal effects of polymyxin-aminoglycoside combination antibiotics on clinical pathogenic bacteria. The conditions for cell culture, treatment, and survival assays were consistent with those described in FIG. 1A. The representative combination drug comprised 0.9 μg/mL polymyxin B and 16 μg/mL amikacin, with A for Klebsiella pneumoniae clinical isolate 9030; with B for Klebsiella pneumoniae clinical isolate 9182; with C for Escherichia coli clinical isolate 0005; with D for Escherichia coli clinical isolate 0011; with E for Pseudomonas aeruginosa clinical isolate 1128; and with F for Staphylococcus aureus clinical isolate 1115. Each measurement was obtained from at least three independent experiments, with error bars representing the mean±standard deviation. The results show that in different clinical isolates of E. coli and Klebsiella pneumoniae, treatment with the combination antibiotic reduced persister survival levels by 100 to 100,000-fold compared to the amikacin monotherapy group; for Pseudomonas aeruginosa and Staphylococcus aureus, the persister levels decreased by 10-fold after the treatment with combination drugs compared to the monotherapy treatment. These results indicate that polymyxin-aminoglycoside combination antibiotics are more effective than individual antibiotics in clearing persistent bacteria, and are universal in both Gram-negative and Positive bacteria.

### EMBODIMENT 5

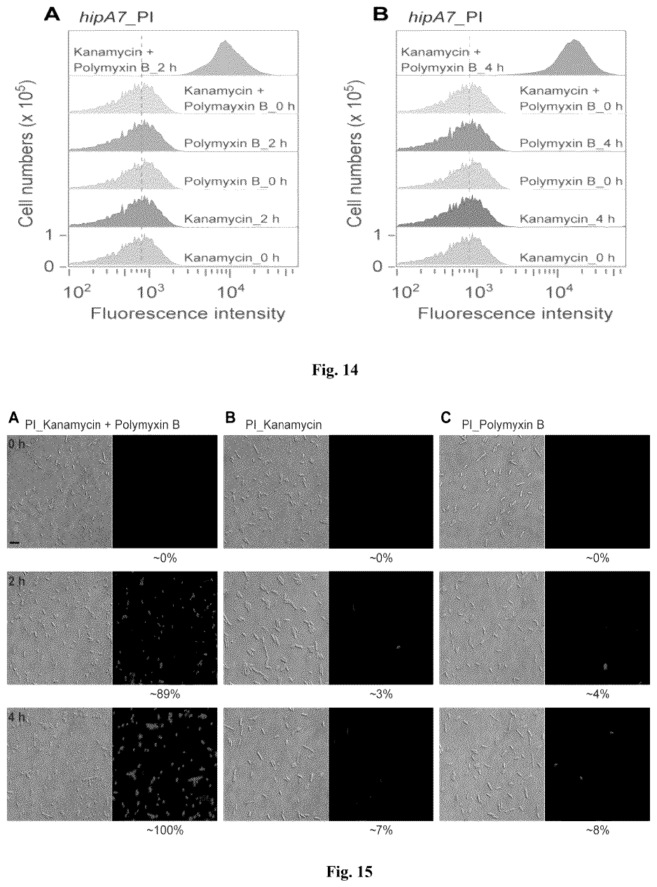

As represented by the combination antibiotic containing 0.9 μg/mL polymyxin B and 20 μg/mL kanamycin, the cell membrane damage in hipA7 cultures during stress-induced antibiotic killing was assessed. As shown in FIG. 13, the polymyxin B-kanamycin combination induced membrane potential loss in hipA7 cells, with A for the combination antibiotic containing 0.9 μg/mL polymyxin B and 20 μg/mL kanamycin, with B for 20 μg/mL kanamycin alone, and C for 0.9 μg/mL polymyxin B. A stationary-phase culture of E. coli hipA7 was inoculated at a 1:20 ratio into fresh LB liquid medium, followed by immediate addition of a combination antibiotic containing final concentrations of 20 μg/mL kanamycin and 0.9 μg/mL polymyxin B and the parallel samples were treated with corresponding concentrations of a individual drug. Samples were incubated at 37° C. with 160 rpm shaking for 8 hours. During sampling, 2.5 μM DiSC3 (5) was added in the first 10 minutes of each time point, followed by washing to remove the drugs and unbound dye. Flow cytometry was used to measure fluorescence intensity, with 100,000 cells analyzed per sample. The results of three independent experiments were basically the same. Representative data from one of the experiments were selected for plotting. During treatment with the combination antibiotics, when labeled with the membrane potential-sensitive fluorescent dye DiSC3(5), the rapid death of hipA7 cells was accompanied by a swift increase in DiSC3(5) signal, indicating a rapid decline in membrane potential. In contrast, no significant enhancement of DiSC3(5) fluorescence signal was observed even after prolonged exposure to kanamycin or polymyxin B alone. Furthermore, propidium iodide (PI) was used to assess cell membrane damage (see FIGS. 14 and 15). Following the combined administration of polymyxin B and kanamycin, the rapid cell death in hipA7 cultures coincided with a sharp rise in PI fluorescence, suggesting membrane disruption; after 2 hours of treatment with either kanamycin or polymyxin B alone, the majority of cells showed no increase in PI fluorescence, with only a minimal number of cells exhibiting detectable signals after 4 hours.

FIG. 14 illustrates the disruption of hipA7 cell membranes induced by the polymyxin B-kanamycin combination antibiotic, with A for 2-hour and B for 4-hour. Under the conditions in FIG. 12, E. coli hipA7 cultures in the stationary phase were diluted into fresh medium and immediately treated with the polymyxin B-kanamycin combination antibiotics. Parallel samples were treated with corresponding concentrations of individual drugs. Samples were collected at 0, 2, and 4 hours, with the addition of 5 μM PI dye 10 minutes prior to each collection time point. After washing with saline to remove antibiotics and extracellular dyes, cellular fluorescence intensity was analyzed by flow cytometry, with 100,000 cells analyzed per sample. The results from three independent experiments were basically the same. Representative data from one of the experiments were selected for plotting. FIG. 15 demonstrates membrane disruption in hipA7 cells induced by polymyxin B-kanamycin combination antibiotics, with A for combination antibiotics containing 0.9 μg/mL polymyxin B and 20 μg/mL kanamycin; B for 20 μg/mL kanamycin alone; C for 0.9 μg/mL polymyxin B alone. The parameters and conditions for E. coli hipA7 cell culture, antibiotic treatment, and PI staining were all performed as described in FIG. 13. Samples at different time points were washed with physiological saline and then the fluorescence intensity of individual cells was measured under a fluorescence microscope. The results from three independent experiments were basically the same. For each sample, multiple fields of view were analyzed. Representative data from one of the experiments were selected for plotting., with the percentage data representing the proportion of PI-positive dead cells relative to the total population, as determined by a statistical analysis of 1,000 cells randomly selected from multiple fields of view.

### EMBODIMENT 6

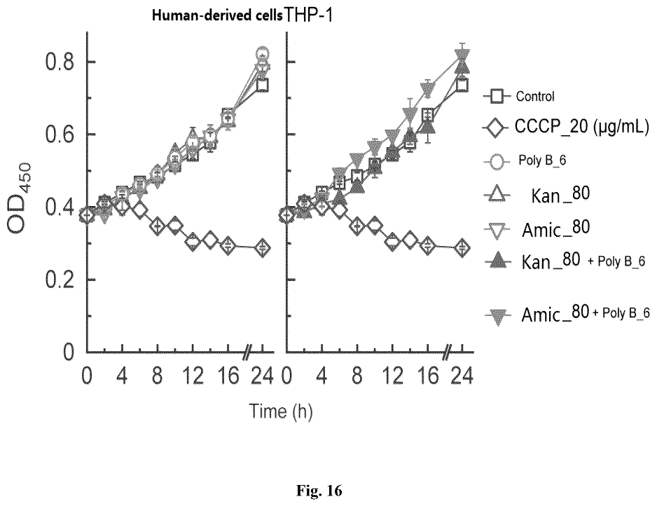

To evaluate the safety of combination antibiotics in human cells, the final concentrations of two components in the combination antibiotics were increased to several times higher than the previously determined bactericidal concentrations. Representative combination antibiotics contained 6 μg/mL polymyxin B and either 80 μg/mL kanamycin or 80 μg/mL amikacin. The effects on the growth and metabolism of human cell line THP-1 were assessed, with cell cultures without the compounds used as blank controls, while those added with CCCP, an inhibitor of the respiratory chain proton motive force used as the positive control. As shown in FIG. 16, the safety profile of polymyxin-aminoglycoside combination antibiotics on human-derived cells was evaluated. THP-1 cells were inoculated in 48-well plates and cultured at 37° C. in a 5% CO₂ incubator for 24 hours, by which time the cell density reached 4˜5×10⁵ cells/mL. Then the combination antibiotics or individual antibiotics at concentrations higher than those in the blood, specifically 80 μg/mL kanamycin, 80 μg/mL amikacin, and 6 μg/mL polymyxin B were added. Parallel samples were treated with 20 μg/mL CCCP as a control. After continuing the culture for 10 hours, the CCK-8 reagent was added at various time points, followed by an additional 1-hour incubation. The OD₄₅₀ values were measured using a microplate reader to assess the impact of high antibiotic concentrations on human cell growth. Each measurement was obtained from at least three independent experiments, with error bars representing the mean±standard deviation. The results indicates that the growth and metabolism of THP-1 cells treated with the two combination antibiotics showed no significant difference compared with the control group without antibiotics. However, CCCP (20 μg/mL) significantly inhibited growth and induced cell death. Therefore, since the peak blood concentrations of the combination antibiotics used for clinical bactericidal purposes are substantially lower than the concentrations applied in this toxicity test, indicating theoretical safety for human cells.

Prior art has not reported any antibiotics that can eliminate nutrient-insensitive persistent bacteria at clinically relevant concentrations, whether used individually or in combination. Regardless of the genotype causing the nutrient-insensitive persistent bacteria—or the initial level of persistence (e.g., 0.001% in wild-type strains versus 5-10% in hipA7 or metG2 strains)—the combination antibiotic therapy of the present invention effectively eradicates such persisters, which demonstrates that the combination antibiotics achieve unexpected technical effects.

1. An application of a combination drug in the preparation of a medicament for treating pathogenic bacterial persistent infections or inhibiting pathogenic bacterial persistence, characterized in that the combination drug comprises a polymyxin antibiotic and an aminoglycoside antibiotic.

2. The application according to claim 1, characterized in that the combination drug is safe for human-derived cells.

3. An application of a combination drug in the preparation of a medicament to enhance the clearance of persistent pathogenic bacteria, characterized in that the combination drug comprises a polymyxin antibiotic and an aminoglycoside antibiotic.

4. The application according to claim 3, characterized in that the drug further comprises other antibacterial agents.

5. The application according to claim 1, characterized in that the pathogenic bacteria are nutrient-insensitive persistent bacteria; the pathogenic bacteria include gram-negative bacteria and gram-positive bacteria.

6. The application according to claim 1, characterized in that, in the combination drug, the weight content of the aminoglycoside antibiotic is 0.01-99.5%.

7. The application according to claim 1, characterized in that the polymyxin antibiotic comprises polymyxin or a salt, hydrate, or solvate thereof; and the aminoglycoside antibiotic comprises an aminoglycoside or a salt, hydrate, or solvate thereof.

8. The application according to claim 7, characterized in that the polymyxin antibiotic comprises polymyxin B or polymyxin E.

9. The application according to claim 7, characterized in that the aminoglycoside antibiotics comprise kanamycin, streptomycin, amikacin, tobramycin, gentamicin, netilmicin, or other similar molecules.

10. The application of a combination drug in the preparation of anti-inflammatory or anti-infective drugs, characterized in that the combination drug comprises polymyxin antibiotic and aminoglycoside antibiotic.
